(19) United States
(12) Patent Application Publication
(10) Pub. No.: US 2020/0080044 A1
(43) Pub. Date: Mar. 12, 2020

(54) PLATE READER, CALIBRATION, SECURE OPERATION, AND IMPROVED PEEL PLATE

(71) Applicant: Charm Sciences, Inc., Lawrence, MA (US)

(72) Inventors: Robert J. Markovsky, Brentwood, NJ (US); Paul E. Graham, Dracut, MA (US); Richard T. Skiffington, North Reading, MA (US); Scott Sutherland, Tewksbury, MA (US)

(73) Assignee: Charm Sciences, Inc., Lawrence, MA (US)

(21) Appl. No.: 16/173,130

(22) PCT Filed: Apr. 28, 2017
(86) PCT No.: PCT/US2017/030030

§ 371 Date: Oct. 29, 2018

Related U.S. Application Data
(60) Provisional application No. 62/362,703, filed on Jul. 15, 2016.
(60) Provisional application No. 62/332,571, filed on May 6, 2016.
(60) Provisional application No. 62/328,665, filed on Apr. 28, 2016.

(51) Int. Cl. C12M 1/34; G01N 15/14; G01N 21/27; G01N 35/00; C12M 1/32

(52) U.S. Cl. CPC C12M 41/36; G01N 15/14; G01N 2015/1486; G01N 35/00594; C12M 23/12; G01N 21/274

(57) ABSTRACT

Reader and plate methods, operations, and systems for imaging/counting biological development on plates are shown and described. One embodiment includes calibrating a plate reader. Calibration may be triggered by detecting a quality control event or the like. The calibration process may include receiving a low and/or high calibrator, counting objects on the calibrator(s), and comparing objects on the calibrator(s) to a predetermined limit. The result is an improved plate reader for observing biological growth, when present, on a growth plate.

This application claims the benefit of PCT application 17/30030, filed Apr. 28, 2017; U.S. provisional application No. 62/328,665, filed Apr. 28, 2016; U.S. provisional application No. 62/332,571, filed May 6, 2016; and U.S. provisional application No. 62/362,703, filed Jul. 15, 2016, all of which are incorporated herein by reference in their entireties.

### FIELD OF THE TECHNOLOGY

The present disclosure relates generally to biological testing, and more particularly to improved methods and operation of plate readers.

### BACKGROUND

It is desirable to provide rapid, effective detection and identification of various and numerous microorganisms in test samples, such as samples of water, food, such as milk, and body fluids. Microorganisms of interest include all aerobic bacteria and specific bacterial groups, such as coliforms. Other microorganisms of interest include a variety of yeast, molds, and the like.

Classical methods for culturing various microorganisms for detection and identification thereof include the spread plate method, the pour plate method and the liquid medium method. In these traditional methods and devices, biological testing is used to identify and quantify the presence of biological matter in samples. Often, these results are used to diagnose biological concerns and begin remedial measures. Particularly in the food industry, where testing is very cost-sensitive, early and accurate diagnosis is desired. In addition, reducing human error is desired, particularly where users might not be laboratory-trained technicians. Tests used must, therefore, be user-friendly and inexpensive without sacrificing accuracy. Further, conventional systems and methods fail to ensure proper and efficient calibration, security measures, and activation of testing devices and procedures.

Therefore, Applicants desire reader calibration and security systems, methods, and quality control devices and operations without the drawbacks presented by the traditional arrangements.

### SUMMARY

In accordance with the present disclosure, methods of calibrating and operating improved plate readers are provided to monitor biological development. This disclosure provides improved methods and devices that are convenient, efficient, and safe for the user, particularly when used to calibrate and activate plate readers to identify biological development, for instance counting microbial colonies, when present.

In one embodiment, a method of negative calibration of a plate reader comprises triggering a calibration activation; inserting a low calibrator within the plate reader; imaging the low calibrator; and counting image counts, wherein counting less than about four image counts, or any predetermined count number, generates a valid determination and counting greater than about four image counts, or any predetermined count number, generates an invalid determination.

In particular examples, inserting the low calibrator includes aligning a low calibrator plate about a sunken support frame. Aligning the low calibrator plate may include positioning a pair of opposing plate proximate apertures in a pair of corresponding proximate frame apertures, and positioning a distal plate platform in a corresponding distal frame platform aperture. A negative calibration check may be triggered on a first count of a startup event. Triggering the calibration check may include detecting an exceeded time period following a previous negative calibration. The time period may comprise about twenty-four hours or the like. Triggering the negative calibration may include detecting an exceeded count limits following a previous calibration check.

The method may include bypassing the triggering of the calibration check. Bypassing may include detecting at least one of the following: detecting at least one previous count following the startup event, detecting less than an exceeded time period following a previous calibration check, and detecting less than an exceeded number of counts following a previous calibration check.

The method typically includes comparing objects on the low calibrator to a predetermined limit. Generating the invalid determination from a number of the detected objects may include substantially greater than a predetermined limit. Further, the method may include activating a cancellation of the calibration, for instance ending a pre-required calibration, upon the invalid determination. Alternatively, the method may include determining a valid result when a number of the detected objects comprise substantially less than a predetermined limit. The method may include activating a completion of the negative calibration, for instance a subsequent required positive calibration check. The method may include prompting an alert to perform a positive calibration. The method may include triggering, for instance automatically, a positive calibration. The method may include prompting a user to align a high calibrator in the plate reader. The method may include comparing objects identified though any of the imaging analysis and procedures herein, on the high calibrator to a predetermined limit. The method may include determining an invalid result when a number of detected objects comprise outside of the predetermined limit.

In certain examples, the reader may generate a calibration determination alert. The method may include generating a no calibration selection. The no calibration selection may be generated by a user identification input. For instance, wherein receiving an administrator identification, a calibration check may be overridden. Alternatively, receiving a non-administrator identification, i.e. a non-proper override identification input, may generate an override denial. The override denial may cancel the calibration check, for instance a required calibration check to activate subsequent sample testing shown and described herein.

In one embodiment, positive calibration of a plate reader includes inserting a high calibrator within the plate reader; imaging the high calibrator; counting image counts identified on the high calibrator; and comparing the image counts to a predetermined positive count, wherein counting a quantity of objects approximate to the predetermined positive count generates a valid determination and counting a quantity of objects outside the predetermined positive count generates an invalid determination.

In particular examples, the method includes selecting manually, either on a technician level or administrator initial setting level, a calibration activation. The approximate positive count may comprise about two percent to about ten percent of the predetermined positive count, for instance the approximate positive count may comprise about five percent of the predetermined positive count.

In certain examples, the method may include prompting an alert to accept the calibration count. Accepting the calibration count may generate a count plate screen. The method may include prompting an alert to reject the calibration count. Rejecting the calibration count may cancel a calibration routine. In some examples, inserting the high calibrator includes aligning a high calibrator plate about a sunken support frame. For instance, aligning the high calibrator plate may include positioning a pair of opposing plate proximate apertures in a pair of corresponding frame proximate apertures, and positioning a distal plate platform in a corresponding distal frame platform aperture.

In another embodiment of the disclosure, in a plate reader for observing biological growth, when present, on a growth plate, a method of calibrating the plate reader comprises selecting a count plate activation; triggering a calibration check; performing a negative calibration; and performing a positive calibration.

In particular examples, selecting the activation includes selecting a count plate button. Triggering the calibration check may include detecting a first count of a startup event. Triggering the calibration check may include detecting an exceeded time period following a previous calibration check. The time period may include about twenty four hours. Triggering the calibration check may include detecting an exceeded limit of counts following a previous calibration check.

In some examples, the method may include bypassing of triggering the calibration check. Bypassing may include detecting at least two events of detecting at least one previous count following the startup event, detecting less than an exceeded time period following a previous calibration check, and detecting less than an exceeded number of counts following a previous calibration check.

In particular examples, the method may include generating an alert to perform the negative calibration. The method may include prompting a user to align a negative calibration plate in the plate reader. Performing the negative calibration may include receiving a negative calibration plate in the plate reader. The method may include counting objects on the negative calibration plate. The method may include comparing objects identified on the negative calibration plate to a predetermined limit. The method may include determining an invalid result when a number of the detected objects comprise substantially greater than the predetermined limit. The method may include activating a cancellation of the calibration check.

In some examples, the method may include determining a valid result when a number of the detected objects comprise substantially less than the predetermined limit. The method may include activating a completion of the calibration check. The method may include prompting a user to perform the positive calibration. The method may include generating an alert to perform the positive calibration. The method may include prompting a user to align a positive calibration plate in the plate reader. Performing the positive calibration may include receiving a positive calibration plate in the plate reader. The method may include counting objects on the positive calibration plate. The method may include comparing objects on the positive calibration plate to a predetermined limit. The method may include determining an invalid result when a number of detected objects comprise substantially greater than the predetermined limit. The method may include cancelling the calibration check. The method may include determining a valid result when a number of detected objects comprise substantially less than the predetelinined limit. The method may include completing the calibration check or cancelling the calibration check.

In particular examples, the method may include generating a calibration determination alert. The method may include generating a no calibration selection. The no calibration selection may generate a user identification input. Receiving an administrator identification input may generate a calibration check override. Receiving a non-administrator identification input may generate an override denial. Further, the override denial may include cancelling the calibration check.

In one embodiment of the disclosure, a peel plate for enumerating a microorganism, when present, in a sample, comprises a recessed well having a sunken wall protruding from an upper face; a pair of opposing proximate extensions adjacent the recessed well; a distal raised platform adjacent the recessed well; and a foldout label aligned between the proximate extensions and the distal raised platform, wherein the foldout label having a collapsible notation tab hinged about a perforated fold.

In particular examples, the foldout label includes a peel tab positioned along a bottom portion of the plate and adapted to removably separate a portion of the label to expose the recessed well. The foldout label may include a hinge securing the notation tab. The notation tab may overlie the foldout label in a stationary position and hinge in an operation position. The notation tab may include a secondary adhesive. The notation tab may include an inner notation face. The notation tab may include an outer notation face.

In some examples, the foldout label has a dimension larger than a dimension of the recessed well. The foldout label may align adjacent the proximate extension and adjacent to the distal raised platform. The recessed well may align below and parallel to an upper face of the plate and may include a grid. The proximate extensions may be spaced between an access indent and adjacent the foldout label. The distal raised platform may span about a length of a diameter of the recessed well. The plate may include a culture medium secured in the recessed well.

In another embodiment of the disclosure, an adhesive cover removably enclosing a growth plate having a recessed well, comprises a foldout label having a securement bottom side and an opposing upper side; an adhesive applied to the bottom side to removably enclose the recessed well; and a tab pivotally secured along one edge of the upper side and having at least one notation face.

In particular examples, the tab aligns above the upper side in a stationary position and pivots substantially parallel and adjacent to the upper side in an operation position. The tab may include a secondary adhesive adapted to releasably secure the tab about the upper side. The tab may include an inner notation face adapted to receive at least one notation. The notation may be a printed notation, a barcode, an electronic coding, a handwritten notation, and any other notation, data, or descriptive identifier. The tab may include an outer notation face receive at least one notation. The tab may include a hinge. The hinge may include a perforation fold. The foldout label may have a dimension larger than a dimension of the recessed well. The growth plate may have at least one proximate extension adjacent the recessed well and a distal raised platform adjacent the recessed well, and the foldout label may align between the proximate extension and the distal raised platform.

In another embodiment of the disclosure, an assembly comprises a growth plate having a recessed well to receive a sample, a raised platform, and a proximate extension; a dried media culture positionable within the recessed well; and a foldout label having a notation tab hingedly secured to the label and having an inner notation face and an outer notation face, and wherein the notation tab overlies the label in a stationary position and hinges to an operation position.

In some examples, the notation tab includes a perforation fold. The notation tab may align parallel to the foldout label's upper side in a stationary position and pivots substantially one hundred and eighty degrees about the perforation fold in the operation position. The notation tab may include a secondary adhesive releasably securing the notation tab about the foldout label in at least one stationary position. The notation tab may include an inner notation face having a substantially flat surface to receive at least one first notation, for instance a printed notation, a barcode, an electronic coding, a handwritten notation, or the like. The notation tab may include an outer notation face having a substantially flat surface to receive a notation. The second notation may be independent of a first notation.

In some examples, the foldout label has a dimension larger than a dimension of the recessed well. The foldout label may align between the proximate extension and the distal raised platform. The foldout label may include a peel tab positionable along a bottom portion of the plate and removably separating a portion of the label to expose the recessed well. The recessed well may align below and parallel to an upper face of the plate and may include a grid. Further, the proximate extensions may be spaced between an access indent and adjacent the foldout label.

The above summary was intended to summarize certain embodiments of the present disclosure. Embodiments will be set forth in more detail in the figures and description of embodiments below. It will be apparent, however, that the description of embodiments is not intended to limit the present inventions, the scope of which should be properly determined by the appended claims.

### BRIEF DESCRIPTION OF THE DRAWINGS

Embodiments of the disclosure will be better understood by a reading of the Description of Embodiments along with a review of the drawings, in which:

FIG. 1 is a front view of one embodiment of a plate reader according to the present disclosure;

FIG. 1A is a front view of one example of loading a plate into the plate reader introduced in FIG. 1;

FIG. 2 is a front view of the plate reader introduced in FIG. 1 in a loaded, operating position;

FIG. 3 is a schematic view of a plate reader assembly according to another embodiment of the disclosure;

FIG. 4 is a perspective view of a plate reader assembly according to one embodiment of the disclosure;

FIG. 5 is a screen view of one embodiment of a plate type selection;

FIG. 5A is a screen shot of a user interface embodiment according to the disclosure;

FIG. 6 is an exploded perspective view of particular system elements;

FIG. 7 is an exploded perspective view of the system shown in FIG. 1 with elements removed for clarity;

FIG. 8 is an isolated, top view of a mounting foundation shown in FIG. 7;

FIG. 9 is an isolated, top perspective view of a base plate shown in FIG. 7;

FIG. 10 is an isolated, top perspective view of an embodiment of a frame nest and corresponding plate;

FIG. 11 is a top perspective view of one embodiment of a peel plate with a singular, planar peel;

FIG. 12 is a top perspective view of one embodiment a peel plate according to the disclosure;

FIG. 13 is a bottom perspective view of the peel plate introduced in FIG. 12;

FIG. 14A is a side perspective view of the peel plate introduced in FIG. 12;

FIG. 14B is a top view of a peel plate embodiment having a foldout label cover;

FIG. 14C is a top perspective view of the example introduced in FIG. 14B, with the peel plate separated from the label cover to illustrate elements;

FIG. 14D is a top perspective view of the example introduced in FIG. 14B, with elements of the foldout label extended in one embodiment of an operation position;

FIG. 15 is an overview flow chart of an operation sequence;

FIG. 16 is a schematic flow chart of one embodiment of a calibration sequence;

FIG. 17 is a schematic flow chart of one embodiment of quality control events triggering a calibration process;

FIG. 18A is a schematic flow chart of one example of a negative calibration sequence;

FIG. 18B is a screen view of one example of a negative calibration prompt;

FIG. 18C is a screen view of one example of a negative calibration result display;

FIG. 19A is a schematic flow chart of one example of a positive calibration sequence;

FIG. 19B is a screen view of one example of a positive calibration result display;

FIG. 20 is a schematic flow chart of another example of a positive calibration sequence;

FIG. 21 is a schematic flow chart of one example of a calibration override process;

FIG. 22 is a schematic flow chart of one embodiment of a calibration sequence;

FIG. 23 is a screen view of one embodiment of a visual check display; and

FIG. 24 is a screen view of another embodiment of a visual check display.

### DESCRIPTION OF EMBODIMENTS

In the following description, like reference characters designate like or corresponding parts throughout the several views. Also in the following description, it is to be understood that such terms as “forward,” “rearward,” “left,” “right,” “upwardly,” “downwardly,” and the like are words of convenience and are not to be construed as limiting terms.

Referring now to the drawings in general, it will be understood that the illustrations are for the purpose of describing embodiments of the disclosure and are not intended to limit the disclosure or any invention thereto. As best seen in the various figures, plate reader systems and assemblies are shown embodied according to the present disclosure for biological growth counting with proper plate seating and activation, increased sample throughput, direct data results reporting, and processed plate image storage. The reader system 10 generally images biological development, when present, on an individual growth plate 20 and/or a plurality of growth plates 20, or similar testing medium. The methods and operation generally include calibration sequences and quality control processes prior to and/or during imaging/counting procedures on any of the plates shown and described herein.

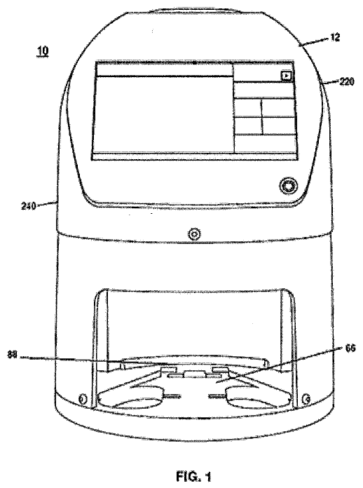

FIGS. 1-2 and 6 illustrate embodiments of a plate reader to generally calibrate the unit before testing, and then receive, image, and count microbial colony growth, when present. These assemblies include plate imaging, processing, and user interface elements, either in electrical communication with one another or combined into an integral assembly, as understood by those of ordinary skill in the art having the benefit of this disclosure.

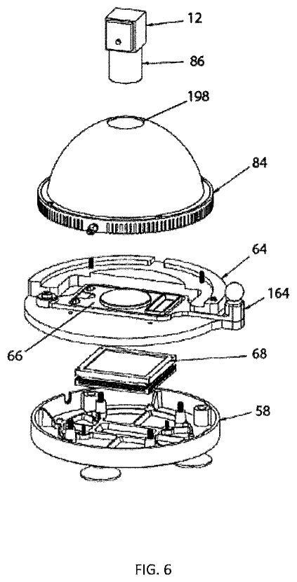

In certain embodiments, housing features and a variety of outer supports align and shield internal components. For instance, as shown in FIG. 1, an outer housing 240 surrounds internal imaging and processing components to generally define an integral system. The housing 240 may include a plurality of openings to allow access to the plate nest frame and like elements shown and described herein. As shown in FIG. 6, useful, although not necessarily required in every embodiment, elements housed within housing 240 may include a mounting foundation 58, a backlight diffuser 68, a base plate 64, a portion of a nest frame 66, an illumination dome 84, and an optics imaging device 12. As shown in FIGS. 4 and 6, the illumination dome 84 may include an optics enclosure 198 to generally enclose the imaging device 12. The illumination dome 84 may evenly illuminate the plates and prevent reflections on the plate surface.

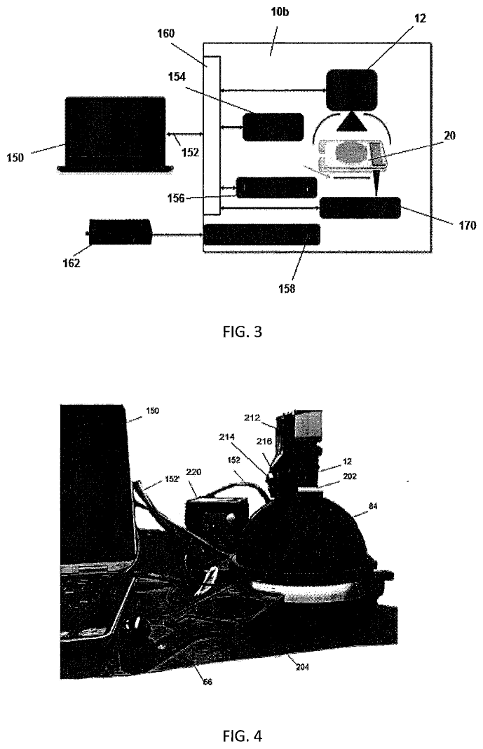

FIG. 3 illustrates one example of a non-integral reader assembly having a plate imaging unit and reader system in data communication with a computer processor 150. The assembly typically includes an image processing engine to perform colony counting to count and/or monitor biological growth, including microbial colony counting, bacterial counting, and the like, when present, on the growth plate. In particular examples, the computer processor 150 is a qualified laptop, tablet, or the like running plate analyzer processing described and shown herein.

As shown in FIGS. 1, 3, and 4, the reader system may include an imaging device 12 adjacent to the plate 20 in an imaging position. An alignment bracket(s), frame, and the like may secure any of elements shown and described herein in a semi-fixed position. For instance, a lower fitting 214 may be affixed to the illumination system, housing, or the like. Similarly, an upper fitting 212 may be affixed to the imaging unit, devices, housing, or the like. The lower fitting 212 and upper fitting 214 may be secured about one another in a variety of configurations and alignments, including, but not limited to, with a fastener 214 or similar linkage. The lower fitting 212 and upper fitting 214 may be positioned together with at least one adjustment. In certain examples, the adjustment includes an off-axis, i.e. a horizontal, vertical, or the similar, adjustment.

The system may include sensors 154 to indicate any of the alignment and/or alert system errors shown and described herein. Further, the system may include illumination control 156 to control any of the illumination elements and aspects herein. In addition, the system may include power distribution 158 to control and distribute power for any of the elements and aspects shown and described herein, and a power supply 162, including but not limited to an external power supply. Certain reader system elements are in electrical communication with a user interface, for instance computer processor 150, via a unified communication interface 160 and/or USB connection 152, 152′ or the like. Those skilled in the art having the benefit of this disclosure will recognize additional orientation of components in electrical communication, including alternative integral and non-integral arrangements of imaging, processing, and display elements herein.

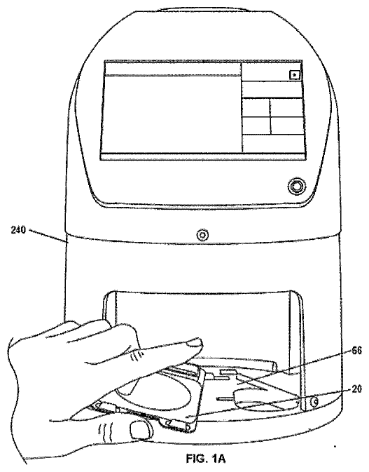

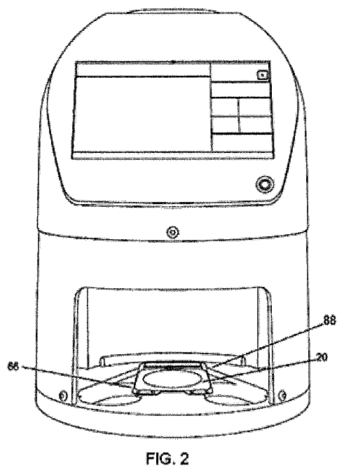

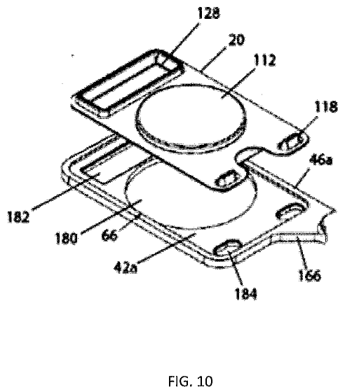

FIG. 10 shows one example of a partially exploded nest frame for illustrating internal alignment components within the assembly as generally shown and described herein. As previously shown in FIGS. 1A and 2, a user may manually load the plate into the nest frame (including aligning any of the growth plate features with any of the frame support features shown and described herein) into a focal alignment with an imaging device in the reader system.

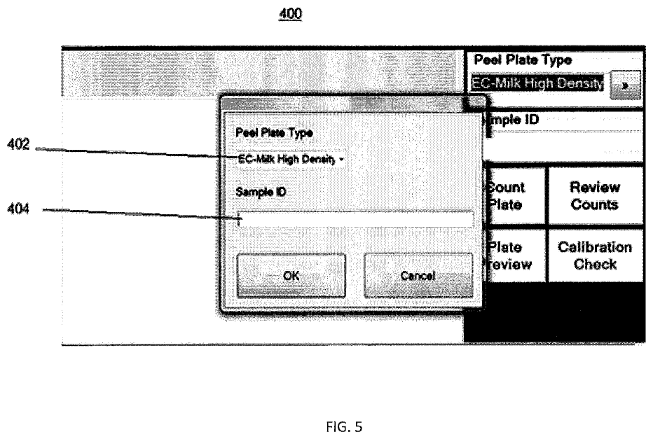

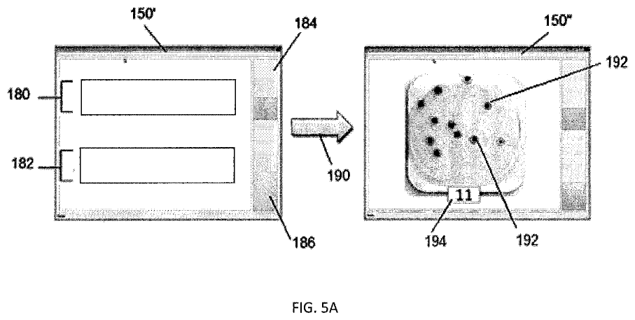

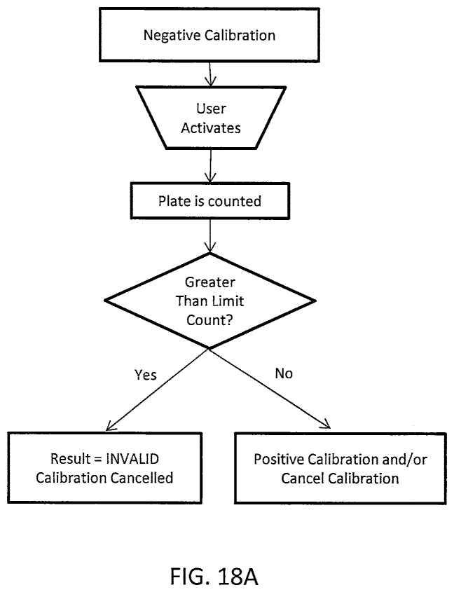

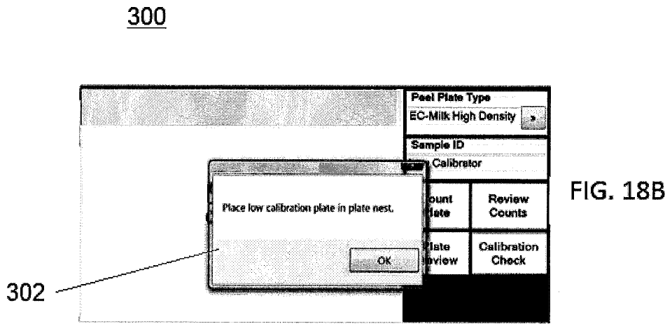

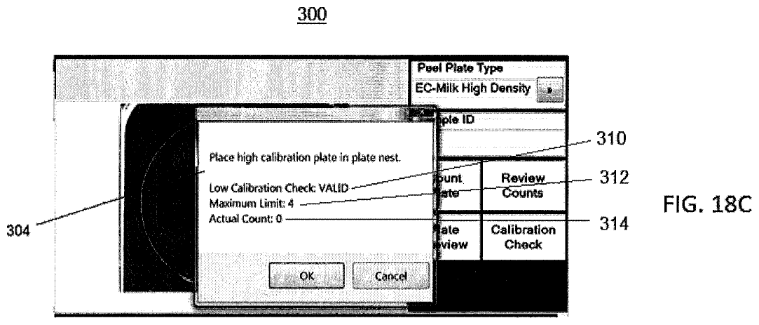

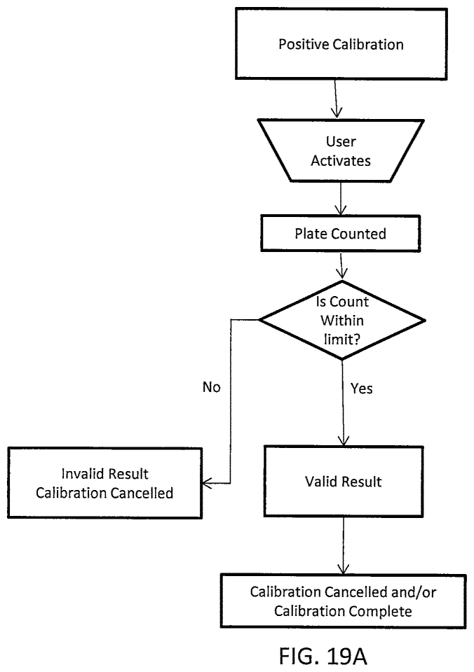

In use, embodiments of the systems and processes may be triggered in a variety of ways, including, but not limited to, manual selection on a user interface, voice activation, remote or timed start, manual positioning of the plate, and the like. In particular examples, the operator manually selects the proper plate and/or count operation to be performed by the assembly, for instance from any variety of selections on a user interface or the like. As illustrated in FIGS. 5, 18B, 18C, embodiments of the user interface selection screen 150′ may include a count plate selection 185, a review counts selection 194, a plate preview selection 195, and a calibration check selection 300, to select and activate any of the systems and processes shown and described herein. Typically the user interface selection screen 150′ includes plate type input 190, for instance chosen from a drop-down selection, button, voice command, manual input, and the like. The selection may include at least a first plate type identifier 180 and a second plate type identifier 182. In other examples, the selection screen 150′ includes a plurality of plate type selection identifiers, including four or more identifiers. The operator may manually select the plate type selection, for instance via clicking, touching, speaking, or the like, the proper icon, voice activating the assembly to types of plates to count, or similar selection processes. The user interface may include a first plate count input selection 184 and a second plate count input selection 186. Again, other examples of the selection screen 150′ includes a plurality of other manual plate type count input selections.

In particular examples, the first plate type identifier 180 includes an aerobic count used for the detection and enumeration of aerobic bacteria in dairy and food decimal dilutions. The aerobic count may include lighting settings, imaging settings, and similar counting settings as recognized by those skilled in the art having the benefit of this disclosure. The second plate type identifier 182 may include an E-coli and coliform count used for detection and enumeration of coliform bacteria, including E-coli in dairy, food, and water. Again, the E-coli and coliform count may include lighting settings, imaging settings, and similar counting settings. Another plate type identifier may include a yeast and mold count for detection and enumeration of yeasts and/or molds in foods and environment. In addition, another plate type identifier may include a heterotrophic plate count used for detection and enumeration of water samples.

In certain examples, a user selects a plate type (including any of the plate type selections shown and described herein). The user may load a blank plate for quality control assurance, and/or calibration as described herein. The device may then capture an image of the blank plate. In particular embodiments, the user interface selection screen 150′ may have a plate preview selection, for instance an image of the plate without performing a count for visual preview and review as understood by those skilled in the art having the benefit of this disclosure.

In certain examples, the image is stored on a storage device, processor, cloud storage, hard drive, or the similar means. In particular embodiments, when a memory, or the like, is approaching eighty five percent, or similar percentage greater than or less than eighty-five percent, capacity a message may be generated to prompt the user to achieve the data. In certain examples, the archival of the data is a manual operation.

In addition, any of the data herein may be secured by limiting access to the folder where the data is saved, for instance at the Windows level, to allow an administrator ensure the folders are invisible and/or not deleteable. In these examples, an end user may disallow a particular user from accessing data at a particular location as understood by those skilled in the art having the benefit of this disclosure.

In certain examples, a user manually loads the plate with the sample and selects the count plate indicator to initiate a particular sequence, for instance the plate is typically manually loaded and the indicator is selected prior to imaging the plate. The imaging device may capture one, or multiple frames averaged together for greater consistency, to create an image using pixel-to-pixel averages for noise reduction of frames. In some examples, the device may set a plate identification, for instance by reading barcode or the like. The system may verify a plate diameter to ensure a proper plate is being analyzed. In particular examples, the system checks the diameter of the plate to verify a proper plate is seated in the system, including, but not limited to, monitoring if the plate is properly seated and an edge is visible to trigger an out of position message, for instance to reject analysis of a particular plate(s).

In particular examples when the second plate type identifier 182 for an E-coli and coliform count is selected, the system loads average and background images. The system may then crop an average image to yield an image of active portions of the pate as recognized by those skilled in the art having the benefit of this disclosure. The system may then crop background image, divide the average image by the background image to yield background-subtracted image. The system may then invert the image and threshold the image in any of the methods shown and described herein, to identify primary objects, including colonies. The image may then be cropped again, and the color objects may be unmixed. For instance in the E-coli and coliform count, the system separates (unmixes and the like) and counts the red color counts and the blue color counts. In particular examples, the results are recorded and saved to a database by any of the procedures described herein.

Similarly, when a first plate type identifier 180 for an aerobic count used for the detection and enumeration of aerobic bacteria is selected, the system loads average and background images. The system may then crop an average image to yield an image of active portions of the pate as recognized by those skilled in the art having the benefit of this disclosure. The system may then crop background image, divide the average image by the background image to yield background-subtracted image. The system may then mask colors of the imagery, typically the mask may be defined in the graphical user interface. The color objects may be unmixed. The system then thresholds the image in any of the methods shown and described herein to identify primary objects, including colonies. In particular examples, the results are recorded and saved to a database by any of the procedures described herein. Those skilled in the art will recognize additional operations and methods, including any image counting method, triggered by a selected plate type selection 190 with the benefit of this disclosure.

In certain embodiments, any of the background and associated values, counts, etc. herein may include a password protection, for instance at an administrator level. For example, any of the “set background” steps or processes may include a login prompt, wherein an administrator level entry allows a background to be specified and an operator level entry is denied setting a particular background process.

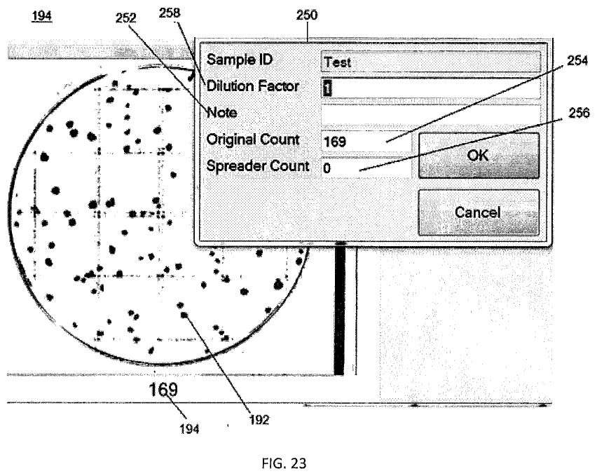

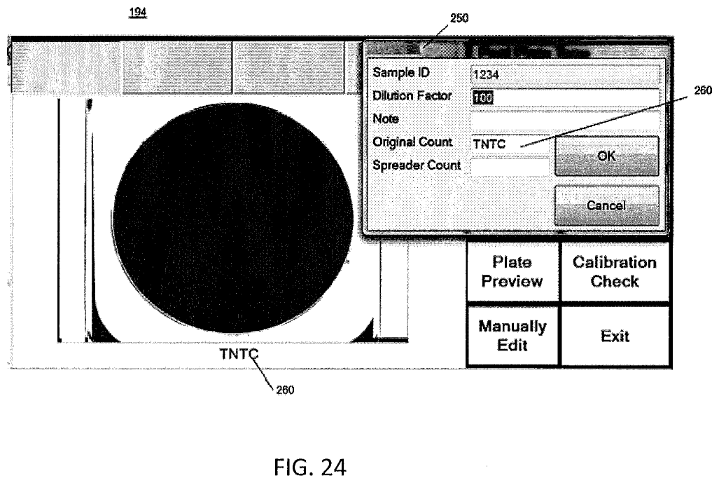

In particular examples, the user interface display 150″ presents a count result 194. FIG. 23 illustrates one example of a visual check 250, wherein the user interface shows a dilution factor 258, notes entry 252, a count result 254 and/or image with counts 192, a spreader count result 256, and the like. Typically, a visual check window may be closed and re-accessed by a manual edit selection, for instance with may be generated under the plate review selection as shown and described herein. A mouse or figure selection may enlarge the image to better view detected and counted colonies. In some examples, each counted colony may have a line encircling the results that may be viewed via enlarging the image. In particular examples, an aerobic count may have a black line, while coliform and the like may generate a colored, for instance red or blue, line depending on the reader settings. As described herein, the counts may be manually edited, for instance in the save results window in the user interface, to determine if there are colony count omissions, additions, amendments, or the like. Further, result notes 252 may be added to any of the results/display herein. FIG. 25 illustrates another example of a visual check 250 with a generated result of a too numerous to count (TNTC) 260 result. In particular examples, the visual check may be generated and suggest a number of colonies and spreaders or a TNTC interpretation. In particular examples, these results may be manually edited and/or stored as shown and described herein.

One embodiment of the plate type selection includes manual entry, for instance a forced sample entry 400 as illustrated in FIG. 5. As shown in FIG. 5, a sample entry 400 on the user interface may include plate type selection 402 and a sample identification entry 404, or the like. The user interface display result may include marking bacterial colonies in a variety of displays, configurations, arrangements, and the like. For instance, as illustrated in FIG. 5A, the display 150″ may present circled bacterial colony counts 192 on an image of the plate, or the like. The processed image 150″ may include a coded name, for instance marked on a barcode or the like as described herein, and a CSV file with corresponding colony count information. The output image and an output report will vary depending on the type of plate being processed. For example, an aerobic count may indicate a unified count of all colonies, whereas an E-coli count contain color categorized colonies.

FIG. 6 illustrates useful internal elements, for instance base plate 64 and mounting foundation 58 assembly. The mounting foundation may include one or a plurality of supports 138, including suction cups, fittings, braces, and the like, to support any of the plate imaging units shown and described herein about a flat surface or similar laboratory bench. Fasteners 139, 156, and 148, as well as grommet 152 and spring plunger 154 may secure the base plate 64 about the mounting foundation 58 and/or other bodies. Further, a backlight diffuser, for instance the backlight box 68 may be positioned between the base plate 64 and mounting foundation 58 to generally diffuse flat lighting under the plate to enhance silhouette detection.

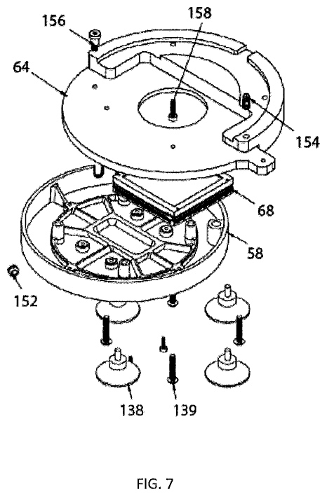

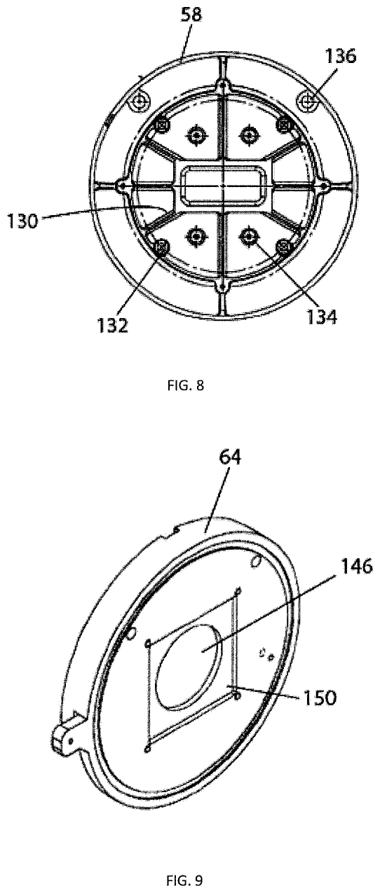

FIG. 7 shows one example of mounting foundation 58 of the image station for supporting the plate imaging unit. The mounting foundation 58 may include one or more mounting holes 136 to mate with the base plate 64. Further, the mounting foundation 58 may include a foundation framing 130, or similar solid supporting, to support the load of any of the elements and examples shown and described herein. The mounting foundation 58 may include base plate couplers 132 and backlight diffuser couplers 134 so support and provide clearance for foundation and lighting elements. Those of ordinary skill in the art having the benefit of this disclosure will recognize additional framing and support elements and alternatives.

FIG. 6 illustrates one example of a baseplate 64 to generally align plate frame nests. For instance, any of the reader devices herein may include a frame nest support to generally receive and retain any of the plates during operation and the like. As shown in FIG. 10, a receiving nest may include a sunken support frame 42a surrounded by a raised boundary 46a, thereby providing a cavity to receive and retain the plates. In particular examples, the support frame 42a may include a recessed distal platform aperture 182, a recessed well aperture 180, and a pair of opposing proximate apertures 184 to mate with a corresponding inverted growth plate's recessed well, pair of opposing proximate extensions, and distal raised platform as shown and described herein. An attachment portion 166 may align the frame nest about any internal structure, for instance within the housing. Further, an optics aperture 146 may be aligned in the alignment cradle 140. In one example, a backlight indent 150 mates with the backlight 68 centered on the optics aperture 146.

Any of the reader devices herein may include a frame nest support to generally receive and retain any of the plates during operation and the like. As shown in FIG. 10, a receiving nest may include a sunken support frame 42a surrounded by a raised boundary 46a, thereby providing a cavity to receive and retain the plates. In particular examples, the support frame 42a may include a recessed distal platform aperture 182, a recessed well aperture 180, and a pair of opposing proximate apertures 184 to mate with a corresponding inverted growth plate's recessed well, pair of opposing proximate extensions, and distal raised platform as shown and described herein. An attachment portion 166 may align the frame nest about imaging elements, for instance within the housing.

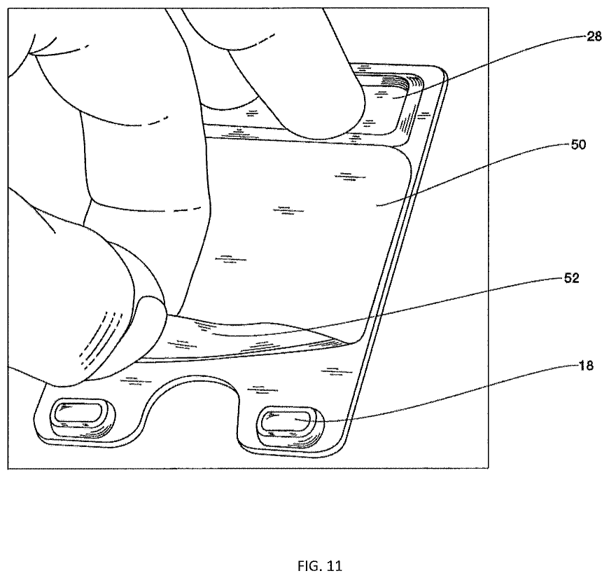

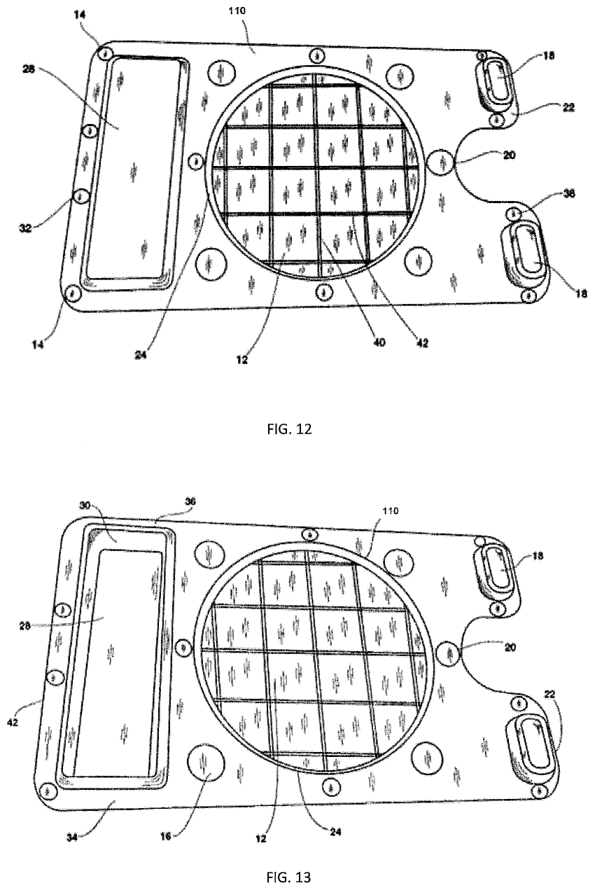

Those of ordinary skill in the art having the benefit of this disclosure will recognize that any of the growth plates shown and described herein may include plate-like devices, Petri dish culture devices, and the like. Typically, the growth plate 20 includes a growth area where biological growth, or the like, may develop. As shown in FIGS. 12-14D, the growth area may be transparent and may have a recessed well that is useful for culturing various microorganisms.

FIG. 11 introduces one example of a peel plate 110 having a covered surface as shown and described herein. For instance, the peel plate 110 may be placed on a substantially level surface. The peel tab 52 may be lifted concurrently while pressure is applied to the raised platform 28 with the user's fingers, or the like. In particular examples, the tab 52 may be lifted vertically upwards and away to expose any of the culture media shown and incorporated herein. In particular the culture media is any of the dried media culture disc shown and described herein.

FIG. 12 introduces one example of a culture device peel plate 110 for enumerating and/or detecting a microorganism from a sample that is useful for the reader examples and embodiments shown and described herein. The peel plate 110 typically is a semi-rigid waterproof plate onto which sample may be applied to enumerate microorganisms and the like. As seen in FIG. 12, one example of the peel plate 110 includes a recessed well 12, a distal raised platform 28, and opposing proximate tabs 22 having proximate extensions 28 to support stacked plates as shown and described herein. The upper face 14 of the plate typically has a top periphery 32 around the raised platform. The recessed well 12 includes a sunken wall 24 below the upper face 14. As shown in FIG. 12, the recessed well may include a grid, for instance having vertical line 40 and intersecting horizontal line 42 components useful for colony counting. In particular examples, the grid is molded, printed, and the like on the rear surface. The grid may be printed in a variety of ways, including inkjet printing, pad printing and the like. Regardless of the grid type, the grid is typically visible through the generally transparent culture device to the front surface and/or rear surface. The plate 110 is also typically transparent material so as to enable observation from the outside, including any of the printed grids shown and described herein.

FIG. 12 further shows the proximate end of the peel plate 110 includes an access indent 20 with opposing proximate tabs 22 between rounded corners 38. Typically, the proximate tabs 22 offset the proximate extensions, and the like, from the body of the plate, i.e. the well and the majority of the upper surface. Thereby the proximate tabs include proximate extensions 18 for alignment, stability, and support during testing/usage, including, but not limited to, layering and stacking plates in any of the arrangements and orientations shown and described.

FIG. 13 shows a bottom and side view, respectively, of one example of a peel plate 110 having a raised edge 30 extending above the lower face 16 to define the raised platform 28. Typically, the peel plate has a distal thickness 42 to support any of the elements and testing procedures shown and described herein.

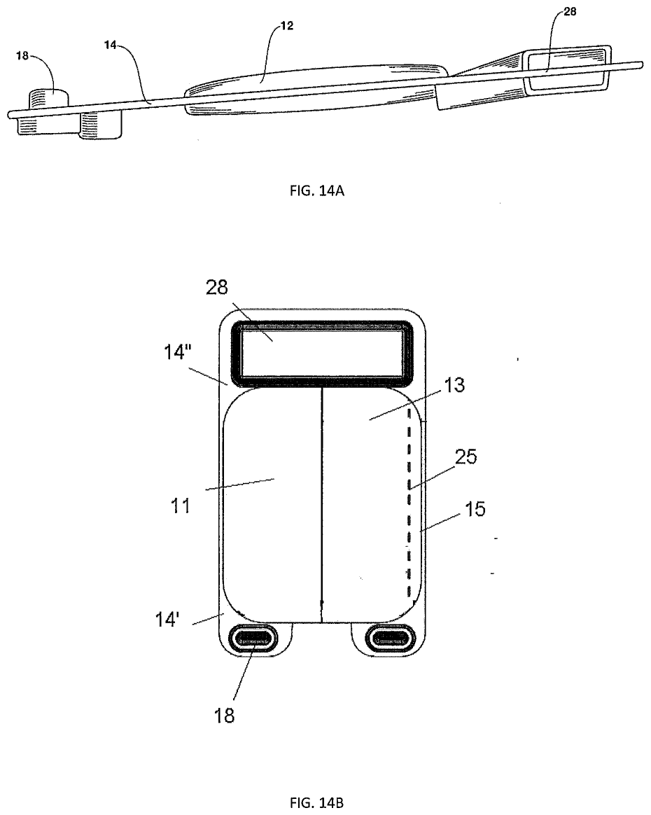

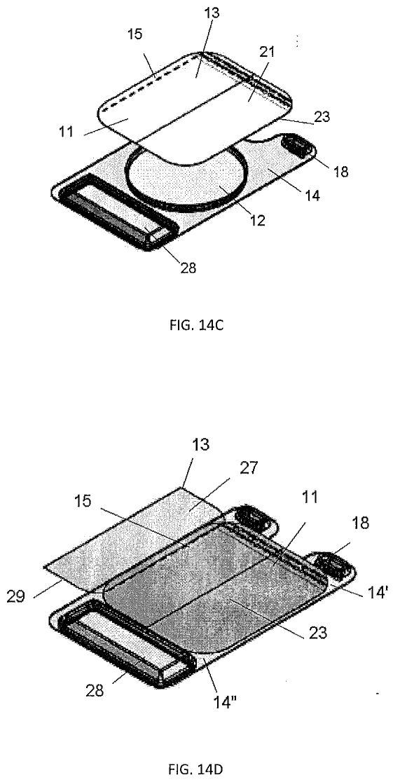

FIG. 14B illustrates one example of the culture device peel plate having a foldout label cover 11 secured about upper portions 14′, 14″. As shown, the foldout label cover 11 is positioned over the recessed well 12 to provide any of the features shown and described herein. Other examples include alternative positioning of the recessed well 12, for instance a reverse alignment, scaled down or scaled up embodiments, offset alignment, and the like. Additional examples may include multiple label covers. Typically, the foldout label 11 aligns between the distal raised platform 28 and at least one proximate extension 18, including adjacent to the distal raised platform 28 and at least one proximate extension 18. However, alternative examples include aligning the foldout label 11 on a peel plate without a distal raised platform 28 and/or a proximate extension 18. The label cover 11 may include a hinge 15 supporting a notation tab 13. Those of ordinary skill in the art having the benefit of this disclosure will recognize many notations suitable for the present inventions. For instance, any of the notations herein may be a printed notation, a barcode, an electronic coding, a handwritten notation, and any other notation, data, or descriptive identifier. Applicant has unexpectedly discovered a larger footprint surface area without sacrificing the compact and storage features of the plates shown and described herein, for instance to support large labels, barcodes, and the like.

In some examples, the foldout label cover 11 may have a perforated fold 25 to allow the notation tab 13 to align above the plate in a stationary position and pivot for narration in any type of operation position (as shown in one example in FIG. 14D). As illustrated in FIG. 14C, the foldout label cover 11 may include a securement bottom side 23 and an opposing upper side 21. As shown and described herein, the securement bottom side 23 may be adhered to the plate surface 14 by any means, including, but not limited to, adhesives, mechanical bonding, fasteners, and the like. In particular examples, as shown in FIG. 14B, portions of the securement bottom side may be permanently affixed to the plate adjacent plate surface 14″, while portions of the securement bottom side may be removably affixed to the plate adjacent plate surface 14′ to provide access to the recessed well as shown and described herein.

Notation tab 13 may be pivotally secured along one edge of the upper side of the foldout label cover 11 and has at least one notation face. The notation tab 13 may be aligned on either edge of label cover 11, for instance to improve right-handed or left-handed user orientation. Further, multiple notation tabs 13 may be aligned on either or both edges of foldout label cover 11 to provide any testing or laboratory best practices as recognized by those skilled in the art having the benefit of this disclosure.

As shown in FIG. 14D, the notation tab 13 may have an inner notation face 27 to receive at least one notation. The notation tab 13 may pivot, fold, or otherwise traverse substantially one-hundred-and-eighty degrees, or any other degree of rotation greater or less than one-hundred-and-eighty degrees for a desired positioning, about perforation fold 25 to create an operation position. The inner notation face 27 may also include a secondary adhesive to releasably secure the notation tab 13 about the upper side 21 of the label cover 11 in a stationary position. The secondary adhesive releasably secures the notation tab 13 in a stationary position prior to use, as well as may releasably secure the notation tab 13 in the stationary position after a first use and/or subsequent manipulations. Similarly, the opposing side of the notation tab 13 includes an outer notation face 29 to receive at least one of the notations shown and described herein.

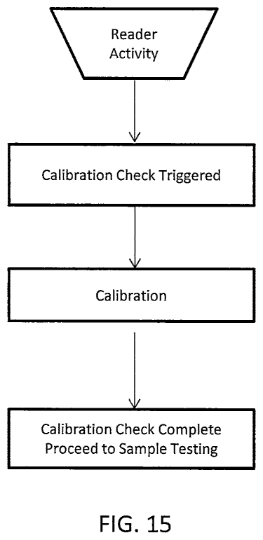

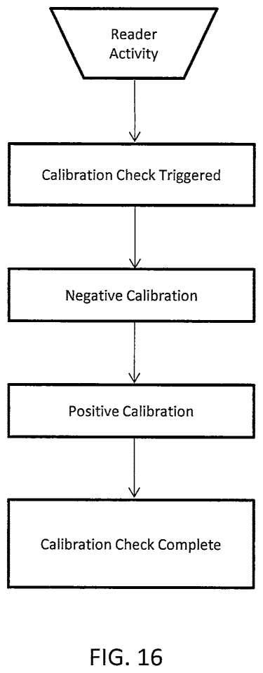

As shown in FIGS. 15, 18B, 18C, and generally described herein, the plate readers, devices, and assemblies may include calibration sequences and quality control processes to improve accurate, reliable test results. FIG. 16 illustrates one example of a calibration sequence 300 that begins with a count plate activation, i.e. any of the activation 190 steps shown and described herein. A calibration sequence 300 may be triggered 306 from a variety of events, and may include a negative calibration 302 and/or a positive calibration 304.

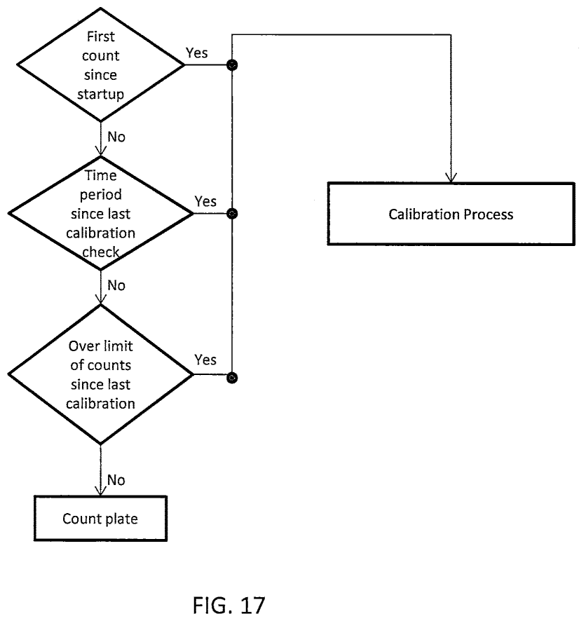

FIG. 17 introduces several events that may trigger 306 a calibration process, while those skilled in the art having the benefit of this disclosure will recognize additional events and procedures steps to trigger calibration or a quality control process. In particular examples, a first count since startup 312 may trigger a calibration process. Thus, detecting a first count of a startup event may trigger the calibration or quality control process. Further, in some examples, a time period since the last calibration check 314 may trigger a calibration process. Thus, detecting an exceeded time period following a previous calibration check may trigger the calibration or quality control process. For instance, the time period may be twenty-four hours, while other examples include greater and less than twenty-four hours. Additionally, in some examples, a number of counts generated by any of the devices and assemblies herein since a last calibration check over a specified limit 316 may trigger a calibration process. Thus, detecting an exceeded limit of counts following a previous calibration check may trigger the calibration or quality control process. As also shown in FIG. 17, in certain examples if no event or situation triggers a calibration process 300, the reader/assembly may count objects in the plate according to any of the examples and embodiments herein.

Certain calibration or quality control processes include a negative calibration, for instance as raised by a “calibration check required now” message, or the like. Procedure steps and processes of one example of a negative calibration 302 sequence is shown in FIG. 18A-18C. A user may activate the system by any of the manual and similar activation steps shown and described; however, alterative examples include automatic activation to initiate any of the calibration sequences or quality control process as recognized by those skilled in the art having the benefit of this disclosure. One example is shown in FIG. 18B, wherein the reader generates a negative calibration prompt 302 to enter a low calibrator, i.e. any of the calibrators shown and described herein. As shown in FIG. 18, the negative calibration may start with a user activation 190, for instance selecting a count plate selection. The plate reader may receive a negative calibration plate, while other examples include utilizing a calibrator that is a non-plate design, for instance without particular plate elements shown and described herein. In particular examples, the system may generate an alert to perform the negative calibration 302. For instance, the process may include prompting a user to align a negative calibration plate in the plate reader, including aligned within the reader nest frame, for a negative calibration plate to be counted 320. The system may count objects 320 on the negative calibration plate and compare the number, size, dimension, color, etc. of the objects to a predetermined limit to determine if the count is greater than the limit 322. In some examples, the determination may generate an INVALID result 324 and a subsequent calibration cancellation, for instance in particular examples, ultimately closing the required calibration for activating any of the sample testing procedures. Alternatively, the determination may generate a valid result 326 and may proceed to a positive calibration 304 and/or a prompt for a positive calibration sequence. FIG. 18C illustrates one example of a negative calibration sequence results 304 display, wherein the VALID result 310, maximum predetermined limit 312, and actual count 314 are generated and displayed on the user interface.

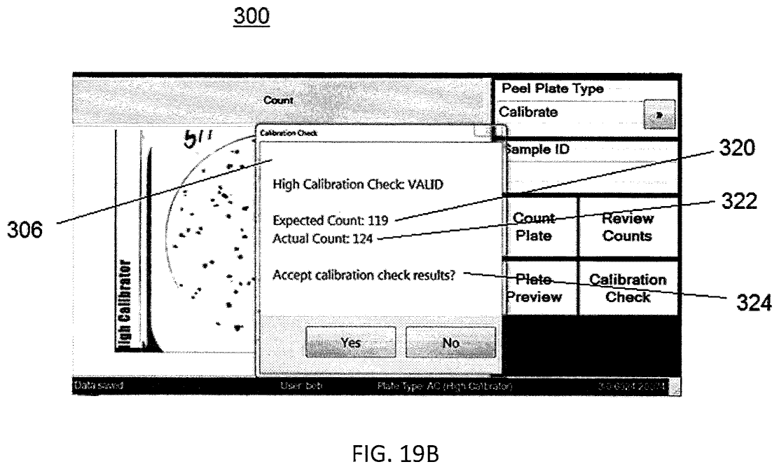

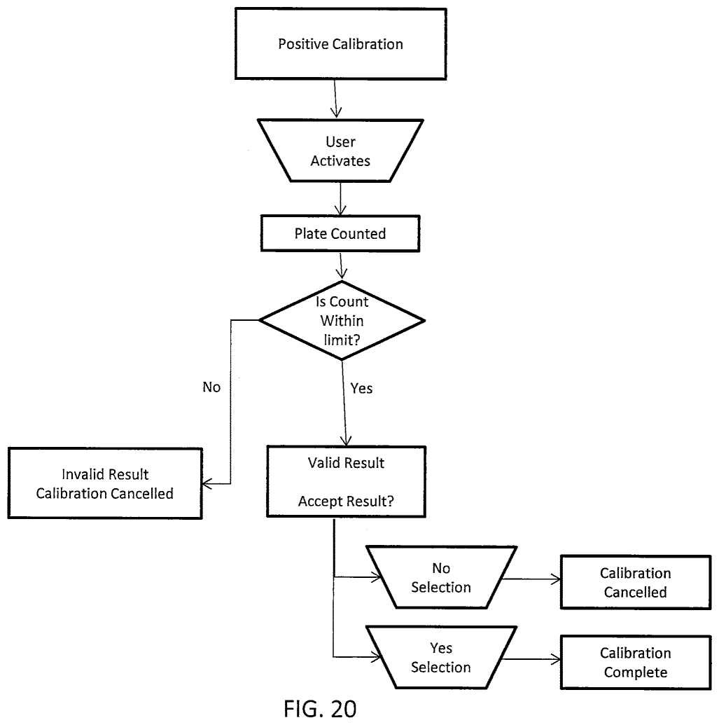

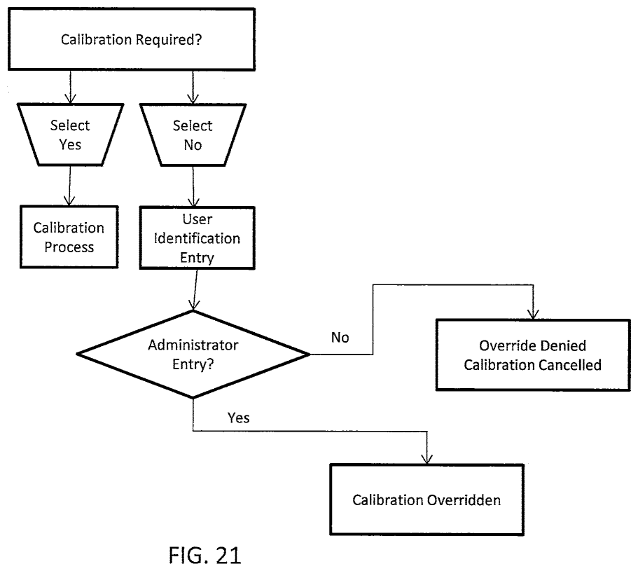

FIGS. 19A, 19B, and 20 introduce embodiments of a positive calibration 304 sequence. A user may activate the system by any of the manual and similar activation steps shown and described; again however, alterative examples include automatic activation to initiate any of the calibration sequences or quality control process as recognized by those skilled in the art having the benefit of this disclosure. The positive calibration may start with a user activation 190, for instance selecting a count plate selection. The plate reader may receive a positive calibration plate, while other examples include utilizing a calibrator that is a non-plate design, for instance without particular plate elements shown and described herein.

In particular examples, the system may generate an alert to perform the positive calibration 304. For instance, the process may include prompting a user to align a positive calibration plate in the plate reader, including aligned within the reader nest frame, for a positive calibration plate to be counted 320. The system may count objects 320 on the positive calibration plate and compare the number, size, dimension, color, etc. of the objects to a predetermined limit to determine if the count is greater than the limit 330.

In some examples, the determination may generate an INVALID result 324 and a calibration cancellation, for instance in particular examples, ultimately closing the required calibration for activating any of the sample testing procedures. FIG. 19B illustrates one example of a positive calibration results 306 display, wherein the VALID result, maximum predetermined expected count 320, actual count 124 are generated, and in certain embodiments, displayed on the user interface. Further, a selection to accept the calibration result 324 may be generated from the sequence and presented on the calibration display 300. Alternatively, the determination may generate a valid result 328 and/or prompt an alert for a calibration complete indication 310. However, as shown in FIG. 20, a valid result 328 may trigger a NO selection acceptance 332 of the result or a YES selection acceptance 334 of the result. For instance, the system may prompt a user to indicate a NO selection 332 or a YES selection 334 regarding the valid result.

In certain examples, the NO selection cancels the calibration sequence 324. While, a YES selection may complete the calibration sequence 310. In particular examples, a complete calibration 310 may set a timer for continued, including semi-continuous, usage without requiring a calibration sequence. For instance, a complete calibration 310 may trigger a twenty-four hour calibration complete timer, while other examples include less than and greater than twenty-four hour periods.

Any of the calibration sequences and quality controls herein may be overridden for a variety of reasons as recognized by those skilled in the art having the benefit of this disclosure. For instance, the system may generate a calibration check required, CHECK NOW? alert, or similar message 308. The system may generate a select YES indication 340 to trigger any of the calibration processes 300 herein. However, the system may also generate a select NO indication 342 and accept user indemnification input 344. For instance, a user may be prompted to enter a username, password, and the like. The system may review the user indemnification to determine an administrator entry 346 or similar properly logged-in user to override any of the calibration sequences and quality controls. In certain examples, when the system receives a valid or proper administrator identification input, a calibration check override 348 may be generated to cancel calibration. Alternatively, when the system receives an invalid or improper administrator identification input, an override denial 350 may be generated and thus requiring any of the calibration and/or quality control processes before generating any of the test results shown and described herein.

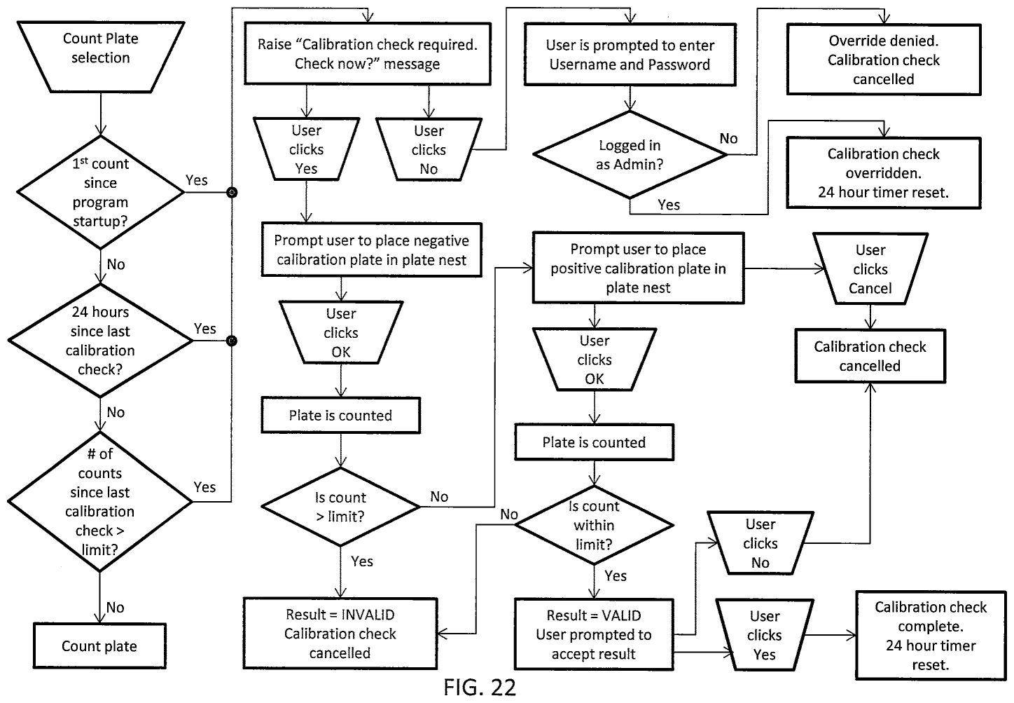

FIG. 22 illustrates additional elements and examples of calibration sequences and quality control processes to improve accurate, reliable test results.

The processors described herein are typically in electrical communication, including USB connection, wireless, or the like, with the plate imaging unit. The processor may include an image processing engine to perform colony counting operations and the like. In particular examples, the image processing engine has image inputs and pipeline parameter inputs. Particular parameter inputs are determined by calibration, including any of the calibration steps and examples herein. Other fixed plate type parameters may be fixed. The image processing engine may generate a variety of outputs, for instance colony counting information.

Further, in alternative embodiments an imaging device may be aligned above the illumination system substantially surrounding the growth plate. The optics may be any of the imaging devices shown and described herein, including a camera to capture any of the still and video images supported by optics communication.

In some examples, the camera includes a moveable lens to manipulate the focal distance of the imaging device to capture a variety of pixel mappings. For instance, the camera lens may be moved closer to the plate or more distant from the plate to gather a variety of pixel mappings, depending on the particular testing sequence.

In some examples, the illumination system includes a plurality of light emitting diodes (LEDs), for instance ninety six, or the like, white LEDs. The light box may include a perimeter lighting frame having a first, second, third and fourth light sides to provide focused light on the top and sides of the peel plate. Further, the light box may include a diffuser.

In yet another alternative example, an imaging device is positioned on the mounting arm about the upper face of the housing. Those of ordinary skill in the art having the benefit of this disclosure will recognize the imaging device may include any optics electronics processing board. Further, the reader may include a processor to provide any of the imaging and analysis shown and described herein.

The vision system for any of the imaging devices shown and descried herein may utilize a grid, reference lines, markings, quadrants, and the like for consistent mapping of specified locations on and among the plates. Further, any of the imaging devices may gather pixel mapping data or values from the entire growth plate or any of the subsections shown and described herein.

In yet other embodiments, several imaging devices may be positioned throughout the reader for generating any of the images show and described herein at a variety of angles with respect to the growth plates. For instance, in some examples the reader may include at least a top and a bottom imaging device, while in other examples the reader may include one mobile imaging device that is capable of moving around, or within, the reader to capture images/scans from the top and bottom perspectives of the growth plates.

In use, the plate imaging unit may be a dynamic tool for monitoring biological agents and development on growth plates, or similar mediums. Generally, the reader system includes imaging technology for observing and quantifying biological growth, when present. In this way, Applicants have unexpectedly discovered the systems shown and described herein enhance the ability to observe changes in the plate development earlier than provided for in conventional systems. Further, the systems and methods herein predict a final result before the final result is actually visible by the human eye. For instance, the systems and methods herein are more sensitive than the human eye and conventional assemblies. In addition, the systems and methods herein monitor the growth plate to find variability prior to test development. For instance, the systems and methods herein establish a more accurate baseline for measuring changes in the growth plate than provided for in the conventional assemblies.

In use, the preliminary image may be first captured with any of the imaging devices shown and described herein under an install calibration. In one example, the settings that configure any optical system for ideal image capture may be predefined during the installation calibration phase of system installation. For instance, optimization of lighting intensity, camera focus and camera exposure time may be defined at an installation calibration set-up. Periodic recalibration may be required due to any of the processing events herein, system aging, and/or metrological conditions. In certain examples, calibration is achieved with pre-printed sample plates, while other examples include non-plate type calibrators having any of the calibration elements shown and described herein.

In one example, mechanical alignment of the growth plate 20 is achieved by drawing a digital circle around the sample area, for instance around a calibration object and/or a microbial colony on a sample testing plate. This digital circle may be manipulated via keyboard, keystrokes to align the circumference and diameter with the sample plate area of interest. Typically, lighting intensity, exposure time, camera focus and mechanical alignment are configuration settings that remain constant after installation, until for instance re-calibration is desired or required.

Further, the image area may be reduced to include only the area of interest that is predefined by the install calibration, subsequent setting, or a by a re-calibration event. Applicants have unexpectantly discovered this reduces processing time, in particular by not having to parse through uninteresting elements, i.e. non-calibration and/or non-microbial colony growth, as understood by those skilled in the art having the benefit of this disclosure.

Numerous characteristics and advantages have been set forth in the foregoing description, together with details of structure and function. Many of the novel features are pointed out in the appended claims. The disclosure, however, is illustrative only, and changes may be made in detail, especially in matters of shape, size, and arrangement of parts, within the principle of the disclosure, to the full extent indicated by the broad general meaning of the terms in which the general claims are expressed. It is further noted that, as used in this application, the singular forms “a,” “an,” and “the” include plural referents unless expressly and unequivocally limited to one referent.

What is claimed is:

1. A method of calibrating a plate reader comprising:
a. detecting at least one event comprising:
i. detecting a first count of a startup event,
ii. detecting an exceeded time period following a previous calibration check, and
iii. detecting an exceeded count limit following a previous calibration check;
b. receiving a negative calibration plate, counting objects identified on said negative calibration plate, and comparing objects on said negative calibration plate to a predetermined limit; and
c. receiving a positive calibration plate, counting objects identified on said positive calibration plate, and comparing objects on said positive calibration plate to a predetermined positive count.

2. The method of claim 1, wherein aligning a calibration plate includes positioning a pair of opposing plate proximate apertures in a pair of corresponding proximate reader frame apertures, and positioning a distal plate platform in a corresponding distal reader frame platform aperture.

3. The method of claim 1, including generating an invalid calibration result when a number of detected objects on said negative calibration plate comprise substantially greater than said predetermined limit.

4. The method of claim 3, wherein counting less than about four image counts on said negative calibration plate generates a valid determination and counting greater than about four image counts generates an invalid determination.

5. The method of claim 4, wherein generating said valid determination includes prompting an alert to perform a positive calibration.

6. The method of claim 3, further including activating a cancellation of said calibration upon said invalid determination.

7. The method of claim 1, wherein counting approximate to said predetermined positive count generates a valid determination and counting outside said predetermined positive count generates an invalid determination.

8. The method of claim 7, wherein said approximate positive count comprises about two percent to about ten percent of said predetermined positive count.

9. The method of claim 1, including generating a calibration determination alert.

10. The method of claim 1, including generating a no calibration administrator user identification input.

11. A method of calibrating a plate reader comprising:
a. counting objects on a first calibrator and comparing objects identified on said first calibrator to a predetermined limit;
b. counting objects on said second calibrator and comparing objects identified on said second calibrator to a predetermined count; and
c. imaging a sample plate and counting bacterial colonies, when present, on said sample plate after objects identified on said first calibrator generate a valid determination and after objects identified on said second calibrator generate a valid determination.

12. The method of claim 11, including aligning said first calibrator about a sunken support frame.

13. The method of claim 11, including aligning said second calibrator about a sunken support frame.

14. The method of claim 12 or claim 13, wherein aligning said calibrator includes positioning a pair of opposing plate proximate apertures in a pair of corresponding proximate frame apertures, and positioning a distal plate platform in a corresponding distal frame platform aperture.

15. A peel plate for enumerating a microorganism, when present, in a sample, said peel plate comprising:
a. a recessed well having a sunken wall protruding from an upper face;
b. a pair of opposing proximate extensions adjacent said recessed well;
c. a distal raised platform adjacent said recessed well; and
d. a foldout label aligned between said proximate extensions and said distal raised platform, wherein said foldout label having a collapsible notation tab hinged about a perforated fold.

16. The device of claim 15, wherein said foldout label includes a peel tab positioned along a bottom portion of said plate and adapted to removably separate a portion of said label to expose said recessed well.

17. The device of claim 15, wherein said notation tab aligns parallel to said foldout label's upper side in a stationary position and pivots substantially one hundred and eighty degrees about said perforation fold in an operation position.

18. The device of claim 15, wherein said notation tab overlies said foldout label in a stationary position and hinges in an operation position.

19. The device of claim 15, wherein said notation tab includes at least one of a group consisting of an inner notation face, an outer notation face, and a secondary adhesive adapted to releaseably secure said notation tab about said foldout label in a stationary position.

20. The device of claim 15, wherein further including a culture medium being secured in a recessed well.
